SEQUENCE LISTING (1 sequence)

SEQ ID NO: 1 - protein, 794 aa, Homo sapiens
MELRPWLLWVVAATGTLVLLAADAQGQKVFTNTWAVRIPGGPAVANSVARKHGFLNLGQIFGDYYHFWHRGVTKRSLSPHRPRHSRLQREPQVQWLEQQVAKRRTKRDVYQEPTDPKFPQQWYLSGVTQRDLNVKAAWAQGYTGHGIVVSILDDGIEKNHPDLAGNYDPGASFDVNDQDPDPQPRYTQMNDNRHGTRCAGEVAAVANNGVCGVGVAYNARIGGVRMLDGEVTDAVEARSLGLNPNHIHIYSASWGPEDDGKTVDGPARLAEEAFFRGVSQGRGGLGSIFVWASGNGGREHDSCNCDGYTNSIYTLSISSATQFGNVPWYSEACSSTLATTYSSGNQNEKQIVTTDLRQKCTESHTGTSASAPLAAGIIALTLEANKNLTWRDMQHLVVQTSKPAHLNANDWATNGVGRKVSHSYGYGLLDAGAMVALAQNWTTVAPQRKCIIDILTEPKDIGKRLEVRKTVTACLGEPNHITRLEHAQARLTLSYNRRGDLAIHLVSPMGTRSTLLAARPHDYSADGFNDWAFMTTHSWDEDPSGEWVLEIENTSEANNYGTLTKFTLVLYGTAPEGLPVPPESSGCKTLTSSQACVVCEEGFSLHQKSCVQHCPPGFAPQVLDTHYSTENDVETIRASVCAPCHASCATCQGPALTDCLSCPSHASLDPVEQTCSRQSQSSRESPPQQQPPRLPPEVEAGQRLRAGLLPSHLPEVVAGLSCAFIVLVFVTVFLVLQLRSGFSFRGVKVYTMDRGLISYKGLPPEAWQEECPSDSEEDEGRGERTAFIKDQSAL

(19) United States
(12) Patent Application Publication
(10) Pub. No.: US 2022/0047556 A1
(43) Pub. Date: Feb. 17, 2022

(54) USE OF SULCONAZOLE AS A FURIN INHIBITOR

(71) Applicants: INSERM (Institut National de la Santé et de la Recherche Médicale), Paris (FR); Université de Bordeaux, Bordeaux (FR); Institut Bergonié, Bordeaux (FR)

(72) Inventors: Abdel-Majid KHATIB, Pessac (FR); Maria Mercedes TOME MONTESINOS, Pessac (FR); Fabienne SOULET, Pessac (FR); Serge EVRARD, Bordeaux (FR); Géraldine SIEGFRIED, Pessac Cedex (FR); Bruno VILLOUTREIX, Lille (FR)

(21) Appl. No.: 17/414,655

(22) PCT Filed: Dec. 16, 2019
(86) PCT No.: PCT/EP2019/085361

§ 371 Date: Jun. 16, 2021

(30) Foreign Application Priority Data
Dec. 17, 2018 (EP) 18306702.4

(51) Int. Cl. A61K 31/4174; A61K 45/06

(52) U.S. Cl. CPC A61K 31/4174; A61K 45/06

(57) ABSTRACT

The proprotein convertases (PCs) are implicated in the activation of various precursor proteins that play a crucial role in various cancers. Using structure-based virtual screening and a compound collection containing FDA approved drugs; the inventors identified Sulconazole as a small molecule able to repress furin activity. Moreover, the inventor show that Sulconazole is particularly suitable for repressing the expression of immune checkpoint protein (e.g. PD-1). 10 Therefore, Sulconazole would be particularly suitable for the treatment of disease involving furin activity in particular for the treatment of cancers.

### FIELD OF THE INVENTION

The present invention relates to use of Sulconazole as a furin inhibitor.

### BACKGROUND OF THE INVENTION

A wide range of proteins involved in intracellular signal pathways require proteolytic cleavage of their protein precursors by the proprotein convertases (PCs) to be biologically active (1). PC family consists of 7 members, namely, furin, PC1, PC2, PC4, PACE4, PC5 and PC7 that convert their unprocessed substrates into functional molecules by cleaving their basic amino acid motifs (K/R)−(X)n−(K/R)↓, where n is 0, 2, 4, or 6 and X is any amino acid (1, 2). These enzymes play an influential role not only in maintaining homeostasis but also in various pathological conditions (1, 2). Various PCs activate proteins involved in malignant transformation and progression including cell surface-expressed receptors, matrix metalloproteinases, growth factors and receptors (1, 2). Altered PC levels were reported to be associated with enhanced invasion and proliferation in various tumor cells. Conversely, inhibition of PC activity by the bioengineered inhibitor, α1-PDX (1, 2), in various cancer cell lines resulted in reduced processing of various PC substrates involved in malignant tumor cells. In a phase I and recent phase II trial (FANG vaccine trial), an autologous tumor-based product targeting furin by shRNAi DNA was found to be beneficial in patients with advanced cancer. The FANG vaccine was safe in patients, and elicited an immune response in patients, which led to prolonged survival (3), suggesting furin expression/activity inhibition as an effective way of boosting antitumor host responses. Thus identification of new inhibitors of proprotein convertase are particularly desirable.

### SUMMARY OF THE INVENTION

The present invention relates to use of Sulconazole as a furin inhibitor. In particular, the present invention is defined by the claims.

### DETAILED DESCRIPTION OF THE INVENTION

The proprotein convertases (PCs) are implicated in the activation of various precursor proteins that play a crucial role in various cancers. Using structure-based virtual screening and a compound collection containing FDA approved drugs; the inventors identified Sulconazole as a small molecule able to repress PCs activity. Moreover, the inventor show that Sulconazole is particularly suitable for repressing the expression of immune checkpoint protein (e.g. PD-1). Therefore, Sulconazole would be particularly suitable for the treatment of disease involving furin activity in particular for the treatment of cancers.

Accordingly, the first object of the present invention relates to the use of Sulconazole as a furin inhibitor.

As used herein, the term “furin” has its general meaning in the art and refers to an enzyme which belongs to the subtilisin-like proprotein convertase (PC) family. The term is also known as paired basic amino acid cleaving enzyme or PACE. Similar to many other proteinases, furin is synthesized as a zymogen (profurin) which becomes active only after the autocatalytic removal of its auto-inhibitory prodomain after its deposition into the appropriate cellular compartment, namely the rough endoplasmic reticulum (RER). The term may include naturally occurring furin and variants and modified forms thereof. The furin can be from any source, but typically is a mammalian (e.g., human and non-human primate) furin, particularly a human furin. An exemplary amino acid sequence for furin is represented by SEQ ID NO:1.

SEQ ID NO: 1
>sp|P09958|FURIN HUMAN Furin
OS = Homo sapiens OX = 9606
GN = FURIN PE = 1 SV = 2
MELRPWLLWVVAATGTLVLLAADAQGQKVF
TNTWAVRIPGGPAVANSVARKHGFLNLGQI
FGDYYHFWHRGVTKRSLSPHRPRHSRLQRE
PQVQWLEQQVAKRRTKRDVYQEPTDPKFPQ
QWYLSGVTQRDLNVKAAWAQGYTGHGIVVS
ILDDGIEKNHPDLAGNYDPGASFDVNDQDP
DPQPRYTQMNDNRHGTRCAGEVAAVANNGV
CGVGVAYNARIGGVRMLDGEVTDAVEARSL
GLNPNHIHIYSASWGPEDDGKTVDGPARLA
EEAFFRGVSQGRGGLGSIFVWASGNGGREH
DSCNCDGYTNSIYTLSISSATQFGNVPWYS
EACSSTLATTYSSGNQNEKQIVTTDLRQKC
TESHTGTSASAPLAAGIIALTLEANKNLTW
RDMQHLVVQTSKPAHLNANDWATNGVGRKV
SHSYGYGLLDAGAMVALAQNWTTVAPQRKC
IIDILTEPKDIGKRLEVRKTVTACLGEPNH
ITRLEHAQARLTLSYNRRGDLAIHLVSPMG
TRSTLLAARPHDYSADGFNDWAFMTTHSWD
EDPSGEWVLEIENTSEANNYGTLTKFTLVL
YGTAPEGLPVPPESSGCKTLTSSQACVVCE
EGFSLHQKSCVQHCPPGFAPQVLDTHYSTE
NDVETIRASVCAPCHASCATCQGPALTDCL
SCPSHASLDPVEQTCSRQSQSSRESPPQQQ
PPRLPPEVEAGQRLRAGLLPSHLPEVVAGL
SCAFIVLVFVTVFLVLQLRSGFSFRGVKVY
TMDRGLISYKGLPPEAWQEECPSDSEEDEG
RGERTAFIKDQSAL

As used herein, the term “furin inhibitor” refers to any compound natural or not which is capable of inhibiting the activity of furin. In particular, the compound of the present invention (i.e. Sulconazole) bind to furin and inhibit its activity.

As used herein, the term “Sulconazole” has its general meaning in the art and refers the compoung having the IUPAC name: 1-[2-[(4-chlorophenyl)methylsulfanyl]-2-(2,4-dichlorophenyl)ethyl]imidazole. The compound is disclosed in U.S. Pat. No. 4,055,652.

The second object of the present invention relates to a method of treating a disease involving furin activity in patient subject in need thereof comprising administering to the patient a therapeutically effective amount of Sulconazole.

As used herein, the term “disease involving furin activity” refers to any disease associated with deleterious effects provoked by furin activity.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

In some embodiments, Sulconazole is suitable for the treatment of autoimmune inflammatory disease. In some embodiments, the autoimmune inflammatory disease is selected from the group consisting of arthritis, rheumatoid arthritis, acute arthritis, chronic rheumatoid arthritis, gouty arthritis, acute gouty arthritis, chronic inflammatory arthritis, degenerative arthritis, infectious arthritis, Lyme arthritis, proliferative arthritis, psoriatic arthritis, vertebral arthritis, and juvenile-onset rheumatoid arthritis, osteoarthritis, arthritis chronica progrediente, arthritis deformans, polyarthritis chronica primaria, reactive arthritis, and ankylosing spondylitis), inflammatory hyperproliferative skin diseases, psoriasis such as plaque psoriasis, gutatte psoriasis, pustular psoriasis, and psoriasis of the nails, dermatitis including contact dermatitis, chronic contact dermatitis, allergic dermatitis, allergic contact dermatitis, dermatitis herpetiformis, and atopic dermatitis, x-linked hyper IgM syndrome, urticaria such as chronic allergic urticaria and chronic idiopathic urticaria, including chronic autoimmune urticaria, polymyositis/dermatomyositis, juvenile dermatomyositis, toxic epidermal necrolysis, scleroderma, systemic scleroderma, sclerosis, systemic sclerosis, multiple sclerosis (MS), spino-optical MS, primary progressive MS (PPMS), relapsing remitting MS (RRMS), progressive systemic sclerosis, atherosclerosis, arteriosclerosis, sclerosis dis seminata, and ataxic sclerosis, inflammatory bowel disease (IBD), Crohn's disease, colitis, ulcerative colitis, colitis ulcerosa, microscopic colitis, collagenous colitis, colitis polyposa, necrotizing enterocolitis, transmural colitis, autoimmune inflammatory bowel disease, pyoderma gangrenosum, erythema nodosum, primary sclerosing cholangitis, episcleritis, respiratory distress syndrome, adult or acute respiratory distress syndrome (ARDS), meningitis, inflammation of all or part of the uvea, iritis, choroiditis, an autoimmune hematological disorder, rheumatoid spondylitis, sudden hearing loss, IgE-mediated diseases such as anaphylaxis and allergic and atopic rhinitis, encephalitis, Rasmussen's encephalitis, limbic and/or brainstem encephalitis, uveitis, anterior uveitis, acute anterior uveitis, granulomatous uveitis, nongranulomatous uveitis, phacoantigenic uveitis, posterior uveitis, autoimmune uveitis, glomerulonephritis (GN), idiopathic membranous GN or idiopathic membranous nephropathy, membrano- or membranous proliferative GN (MPGN), rapidly progressive GN, allergic conditions, autoimmune myocarditis, leukocyte adhesion deficiency, systemic lupus erythematosus (SLE) or systemic lupus erythematodes such as cutaneous SLE, subacute cutaneous lupus erythematosus, neonatal lupus syndrome (NLE), lupus erythematosus disseminatus, lupus (including nephritis, cerebritis, pediatric, non-renal, extra-renal, discoid, alopecia), juvenile onset (Type I) diabetes mellitus, including pediatric insulin-dependent diabetes mellitus (IDDM), adult onset diabetes mellitus (Type II diabetes), autoimmune diabetes, idiopathic diabetes insipidus, immune responses associated with acute and delayed hypersensitivity mediated by cytokines and T-lymphocytes, tuberculosis, sarcoidosis, granulomatosis, lymphomatoid granulomatosis, Wegener's granulomatosis, agranulocytosis, vasculitides, including vasculitis, large vessel vasculitis, polymyalgia rheumatica, giant cell (Takayasu's) arteritis, medium vessel vasculitis, Kawasaki's disease, polyarteritis nodosa, microscopic polyarteritis, CNS vasculitis, necrotizing, cutaneous, hypersensitivity vasculitis, systemic necrotizing vasculitis, and ANCA-associated vasculitis, such as Churg-Strauss vasculitis or syndrome (CSS), temporal arteritis, aplastic anemia, autoimmune aplastic anemia, Coombs positive anemia, Diamond Blackfan anemia, hemolytic anemia or immune hemolytic anemia including autoimmune hemolytic anemia (AIHA), pernicious anemia (anemia perniciosa), Addison's disease, pure red cell anemia or aplasia (PRCA), Factor VIII deficiency, hemophilia A, autoimmune neutropenia, pancytopenia, leukopenia, diseases involving leukocyte diapedesis, CNS inflammatory disorders, multiple organ injury syndrome such as those secondary to septicemia, trauma or hemorrhage, antigen-antibody complex-mediated diseases, anti-glomerular basement membrane disease, anti-phospholipid antibody syndrome, allergic neuritis, Bechet's or Behcet's disease, Castleman's syndrome, Goodpasture's syndrome, Reynaud's syndrome, Sjogren's syndrome, Stevens-Johnson syndrome, pemphigoid such as pemphigoid bullous and skin pemphigoid, pemphigus, optionally pemphigus vulgaris, pemphigus foliaceus, pemphigus mucus-membrane pemphigoid, pemphigus erythematosus, autoimmune polyendocrinopathies, Reiter's disease or syndrome, immune complex nephritis, antibody-mediated nephritis, neuromyelitis optica, polyneuropathies, chronic neuropathy, IgM polyneuropathies, IgM-mediated neuropathy, thrombocytopenia, thrombotic thrombocytopenic purpura (TTP), idiopathic thrombocytopenic purpura (ITP), autoimmune orchitis and oophoritis, primary hypothyroidism, hypoparathyroidism, autoimmune thyroiditis, Hashimoto's disease, chronic thyroiditis (Hashimoto's thyroiditis); subacute thyroiditis, autoimmune thyroid disease, idiopathic hypothyroidism, Grave's disease, polyglandular syndromes such as autoimmune polyglandular syndromes (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis, allergic encephalomyelitis, experimental allergic encephalomyelitis (EAE), myasthenia gravis, thymoma-associated myasthenia gravis, cerebellar degeneration, neuromyotonia, opsoclonus or opsoclonus myoclonus syndrome (OMS), and sensory neuropathy, multifocal motor neuropathy, Sheehan's syndrome, autoimmune hepatitis, chronic hepatitis, lupoid hepatitis, giant cell hepatitis, chronic active hepatitis or autoimmune chronic active hepatitis, lymphoid interstitial pneumonitis, bronchiolitis obliterans (non-transplant) vs NSIP, Guillain-Barre syndrome, Berger's disease (IgA nephropathy), idiopathic IgA nephropathy, linear IgA dermatosis, primary biliary cirrhosis, pneumonocirrhosis, autoimmune enteropathy syndrome, Celiac disease, Coeliac disease, celiac sprue (gluten enteropathy), refractory sprue, idiopathic sprue, cryoglobulinemia, amylotrophic lateral sclerosis (ALS; Lou Gehrig's disease), coronary artery disease, autoimmune ear disease such as autoimmune inner ear disease (AGED), autoimmune hearing loss, opsoclonus myoclonus syndrome (OMS), polychondritis such as refractory or relapsed polychondritis, pulmonary alveolar proteinosis, amyloidosis, scleritis, a non-cancerous lymphocytosis, a primary lymphocytosis, which includes monoclonal B cell lymphocytosis, optionally benign monoclonal gammopathy or monoclonal garnmopathy of undetermined significance, MGUS, peripheral neuropathy, paraneoplastic syndrome, channelopathies such as epilepsy, migraine, arrhythmia, muscular disorders, deafness, blindness, periodic paralysis, and channelopathies of the CNS, autism, inflammatory myopathy, focal segmental glomerulosclerosis (FSGS), endocrine opthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases such as autoimmune demyelinating diseases, diabetic nephropathy, Dressler's syndrome, alopecia greata, CREST syndrome (calcinosis, Raynaud's phenomenon, esophageal dysmotility, sclerodactyl), and telangiectasia), male and female autoimmune infertility, mixed connective tissue disease, Chagas' disease, rheumatic fever, recurrent abortion, farmer's lung, erythema multiforme, post-cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, leprosy, malaria, leishmaniasis, kypanosomiasis, schistosomiasis, ascariasis, aspergillosis, Sampter's syndrome, Caplan's syndrome, dengue, endocarditis, endomyocardial fibrosis, diffuse interstitial pulmonary fibrosis, interstitial lung fibrosis, idiopathic pulmonary fibrosis, cystic fibrosis, endophthalmitis, erythema elevatum et diutinum, erythroblastosis fetalis, eosinophilic faciitis, Shulman's syndrome, Felty's syndrome, flariasis, cyclitis such as chronic cyclitis, heterochronic cyclitis, iridocyclitis, or Fuch's cyclitis, Henoch-Schonlein purpura, human immunodeficiency virus (HIV) infection, echovirus infection, cardiomyopathy, Alzheimer's disease, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis ubiterans, thyrotoxicosis, tabes dorsalis, chorioiditis, giant cell polymyalgia, endocrine ophthamopathy, chronic hypersensitivity pneumonitis, keratoconjunctivitis sicca, epidemic keratoconjunctivitis, idiopathic nephritic syndrome, minimal change nephropathy, benign familial and ischemia-reperfusion injury, retinal autoimmunity, joint inflammation, bronchitis, chronic obstructive airway disease, silicosis, aphthae, aphthous stomatitis, arteriosclerotic disorders, aspermiogenese, autoimmune hemolysis, Boeck's disease, cryoglobulinemia, Dupuytren's contracture, endophthalmia phacoanaphylactica, enteritis allergica, erythema nodosum leprosum, idiopathic facial paralysis, chronic fatigue syndrome, febris rheumatica, Hamman-Rich's disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica, orchitis granulomatosa, pancreatitis, polyradiculitis acuta, pyoderma gangrenosum, Quervain's thyreoiditis, acquired splenic atrophy, infertility due to antispermatozoan antobodies, non-malignant thymoma, vitiligo, SCID and Epstein-Barr virus-associated diseases, acquired immune deficiency syndrome (AIDS), parasitic diseases such as Lesihmania, toxic-shock syndrome, food poisoning, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, sympathetic ophthalmia, rheumatic diseases, mixed connective tissue disease, nephrotic syndrome, insulitis, polyendocrine failure, peripheral neuropathy, autoimmune polyglandular syndrome type I, adult-onset idiopathic hypoparathyroidism (AOIH), alopecia totalis, dilated cardiomyopathy, epidermolisis bullosa acquisita (EBA), hemochromatosis, myocarditis, nephrotic syndrome, primary sclerosing cholangitis, purulent or nonpurulent sinusitis, acute or chronic sinusitis, ethmoid, frontal, maxillary, or sphenoid sinusitis, an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils, anaphylaxis, seronegative spondyloarthritides, polyendocrine autoimmune disease, sclerosing cholangitis, sclera, episclera, chronic mucocutaneous candidiasis, Bruton's syndrome, transient hypogammaglobulinemia of infancy, Wiskott-Aldrich syndrome, ataxia telangiectasia, autoimmune disorders associated with collagen disease, rheumatism, neurological disease, ischemic re-perfusion disorder, reduction in blood pressure response, vascular dysfunction, antgiectasis, tissue injury, cardiovascular ischemia, hyperalgesia, cerebral ischemia, and disease accompanying vascularization, allergic hypersensitivity disorders, glomerulonephritides, reperfusion injury, reperfusion injury of myocardial or other tissues, dermatoses with acute inflammatory components, acute purulent meningitis or other central nervous system inflammatory disorders, ocular and orbital inflammatory disorders, granulocyte transfusion-associated syndromes, cytokine-induced toxicity, acute serious inflammation, chronic intractable inflammation, pyelitis, pneumonocirrhosis, diabetic retinopathy, diabetic large-artery disorder, endarterial hyperplasia, peptic ulcer, valvulitis, and endometriosis.

In some embodiments, Sulconazole is particularly suitable for the treatment of viral infections. In some embodiments, the viral infection comprises infection by one or more viruses selected from the group consisting of Arenaviridae, Astroviridae, Birnaviridae, Bromoviridae, Bunyaviridae, Caliciviridae, Closteroviridae, Comoviridae, Cystoviridae, Flaviviridae, Flexiviridae, Hepevirus, Leviviridae, Luteoviridae, Mononegavirales, Mosaic Viruses, Nidovirales, Nodaviridae, Orthomyxoviridae, Picobirnavirus, Picornaviridae, Potyviridae, Reoviridae, Retroviridae, Sequiviridae, Tenuivirus, Togaviridae, Tombusviridae, Totiviridae, Tymoviridae, Hepadnaviridae, Herpesviridae, Paramyxoviridae or Papillomaviridae viruses. Relevant taxonomic families of RNA viruses include, without limitation, Astroviridae, Birnaviridae, Bromoviridae, Caliciviridae, Closteroviridae, Comoviridae, Cystoviridae, Flaviviridae, Flexiviridae, Hepevirus, Leviviridae, Luteoviridae, Mononegavirales, Mosaic Viruses, Nidovirales, Nodaviridae, Orthomyxoviridae, Picobirnavirus, Picornaviridae, Potyviridae, Reoviridae, Retroviridae, Sequiviridae, Tenuivirus, Togaviridae, Tombusviridae, Totiviridae, and Tymoviridae viruses. In some embodiments, the viral infection comprises infection by one or more viruses selected from the group consisting of adenovirus, rhinovirus, hepatitis, immunodeficiency virus, polio, measles, Ebola, Coxsackie, Rhino, West Nile, small pox, encephalitis, yellow fever, coronavirus, Dengue, influenza (including human, avian, and swine), lassa, lymphocytic choriomeningitis, junin, machuppo, guanarito, hantavirus, Rift Valley Fever, La Crosse, California encephalitis, Crimean-Congo, Marburg, Japanese Encephalitis, Kyasanur Forest, Venezuelan equine encephalitis, Eastern equine encephalitis, Western equine encephalitis, severe acute respiratory syndrome (SARS), parainfluenza, respiratory syncytial, Punta Toro, Tacaribe, pachindae viruses, adenovirus, Dengue fever, influenza A and influenza B (including human, avian, and swine), junin, measles, parainfluenza, Pichinde, punta toro, respiratory syncytial, rhinovirus, Rift Valley Fever, severe acute respiratory syndrome (SARS), Tacaribe, Venezuelan equine encephalitis, West Nile and yellow fever viruses, tick-borne encephalitis virus, Japanese encephalitis virus, St. Louis encephalitis virus, Murray Valley virus, Powassan virus, Rocio virus, louping-ill virus, Banzi virus, Ilheus virus, Kokobera virus, Kunjin virus, Alfuy virus, bovine diarrhea virus, and Kyasanur forest disease.

In some embodiments, Sulconazole is particularly suitable for the treatment of neurodegenerative diseases. Exemplary neurodegenerative diseases include HIV-associated Dementia, multiple sclerosis, Alzheimer's Disease, Parkinson's Disease, amyotrophic lateral sclerosis, and Pick's Disease. As used herein, the term “neurodegenerative disease” shall be taken to mean a disease that is characterized by neuronal cell death. The neuronal cell death observed in a neurodegenerative disease is often preceded by neuronal dysfunction, sometimes by several years. Accordingly, the term “neurodegenerative disease” includes a disease or disorder that is characterized by neuronal dysfunction and eventually neuronal cell death. Often neurodegenerative diseases are also characterized by increased gliosis (e.g., astrocytosis or microgliosis) in the region/s of neuronal death. Cellular events observed in a neurodegenerative disease often manifest as a behavioral change (e.g., deterioration of thinking and/or memory) and/or a movement change (e.g., tremor, ataxia, postural change and/or rigidity). Examples of neurodegenerative disease include, for example, FTLD, amyotrophic lateral sclerosis, ataxia (e.g., spinocerebellar ataxia or Friedreich's Ataxia), Creutzfeldt-Jakob Disease, a polyglutamine disease (e.g., Huntington's disease or spinal bulbar muscular atrophy), Hallervorden-Spatz disease, idiopathic torsion disease, Lewy body disease, multiple system atrophy, neuroanthocytosis syndrome, olivopontocerebellar atrophy, Pelizaeus-Merzbacher disease, progressive supranuclear palsy, syringomyelia, torticollis, spinal muscular atrophy or a trinucleotide repeat disease (e.g., Fragile X Syndrome).

In some embodiments, Sulconazole is particularly suitable for the treatment of cancer. As used herein, the term “cancer” has its general meaning in the art and includes, but is not limited to, solid tumors and blood-borne tumors. The term cancer includes diseases of the skin, tissues, organs, bone, cartilage, blood and vessels. The term “cancer” further encompasses both primary and metastatic cancers. Examples of cancers that may be treated by methods and compositions of the invention include, but are not limited to, cancer cells from the bladder, blood, bone, bone marrow, brain, breast, colon, esophagus, gastrointestinal tract, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; Leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

A further object of the present invention relates to a method of enhancing the proliferation, migration, persistence and/or activity of cytotoxic T lymphocytes (CTLs) in a subject in need thereof comprising administering to the subject a therapeutically effective amount of Sulconazole.

More specifically, the present invention provides a method of therapy in subjects in need thereof, comprising administering to the subject a therapeutically effective amount of Sulconazole that reduces the expression of an immune checkpoint protein, wherein said administration enhances the proliferation, migration, persistence and/or activity of cytotoxic T lymphocytes (CTLs) in the subject.

More particularly, the present invention provides a method of reducing T cell exhaustion in a subject in need thereof comprising administering to the subject a therapeutically effective amount of Sulconazole.

As used herein, the term “cytotoxic T lymphocyte” or “CTL” has its general meaning in the art and refers to a subset of T cells which express CD8 on their surface. CD8 antigens are members of the immunoglobulin supergene family and are associative recognition elements in major histocompatibility complex class I-restricted interactions. They are MHC class I-restricted, and function as cytotoxic T cells. Cytotoxic T lymphocytes are also called, CD8+ T cells, T-killer cells, cytolytic T cells, or killer T cells. The ability of sulconazole to enhance proliferation, migration, persistence and/or cytotoxic activity of cytotoxic T lymphocytes may be determined by any assay well known in the art. Typically said assay is an in vitro assay wherein cytotoxic T lymphocytes are brought into contact with target cells (e.g. target cells that are recognized and/or lysed by cytotoxic T lymphocytes). For example, Sulconazole would be suitable to increase specific lysis by cytotoxic T lymphocytes by more than about 20%, preferably with at least about 30%, at least about 40%, at least about 50%, or more of the specific lysis obtained at the same effector: target cell ratio with cytotoxic T lymphocytes that are contacted by Sulconazole. Examples of protocols for classical cytotoxicity assays are conventional.

As used herein the term “immune checkpoint protein” has its general meaning in the art and refers to a molecule that is expressed by T cells in that either turn up a signal (stimulatory checkpoint molecules) or turn down a signal (inhibitory checkpoint molecules). Immune checkpoint molecules are recognized in the art to constitute immune checkpoint pathways similar to the CTLA-4 and PD-1 dependent pathways (see e.g. Pardoll, 2012. Nature Rev Cancer 12:252-264; Mellman et al., 2011. Nature 480:480-489). Examples of inhibitory checkpoint molecules include B7-H3, B7-H4, BTLA, CTLA-4, CD277, KIR, PD-1, LAG-3, TIM-3, TIGIT and VISTA. B7-H3, also called CD276, was originally understood to be a co-stimulatory molecule but is now regarded as co-inhibitory. B7-H4, also called VTCN1, is expressed by tumor cells and tumor-associated macrophages and plays a role in tumor escape. B and T Lymphocyte Attenuator (BTLA), also called CD272, is a ligand of HVEM (Herpesvirus Entry Mediator). Cell surface expression of BTLA is gradually downregulated during differentiation of human CD8+ T cells from the naive to effector cell phenotype, however tumor-specific human CD8+ T cells express high levels of BTLA. CTLA-4, Cytotoxic T-Lymphocyte-Associated protein 4 and also called CD152 is overexpressed on Treg cells serves to control T cell proliferation. KIR, Killer-cell Immunoglobulin-like Receptor, is a receptor for MHC Class I molecules on Natural Killer cells. LAG3, Lymphocyte Activation Gene-3, works to suppress an immune response by action to Tregs as well as direct effects on CD8+ T cells. TIM-3, short for T-cell Immunoglobulin domain and Mucin domain 3, expresses on activated human CD4+ T cells and regulates Th1 and Th17 cytokines. TIM-3 acts as a negative regulator of Th1/Tc1 function by triggering cell death upon interaction with its ligand, galectin-9. VISTA, short for V-domain Ig suppressor of T cell activation, is primarily expressed on hematopoietic cells so that consistent expression of VISTA on leukocytes within tumors may allow VISTA blockade to be effective across a broad range of solid tumors. As used herein, the term “PD-1” has its general meaning in the art and refers to programmed cell death protein 1 (also known as CD279). PD-1 acts as an immune checkpoint, which upon binding of one of its ligands, PD-L1 or PD-L2, enables Shp2 to dephosphorylate CD28 and inhibits the activation of T cells.

In some embodiments, Sulconazole is particularly suitable for reducing the expression of PD-1.

As used herein, the term “T cell exhaustion” refers to a state of T cell dysfunction. The T cell exhaustion generally arises during many chronic infections and cancer. T cell exhaustion can be defined by poor effector function, sustained expression of inhibitory receptors, and/or a transcriptional state distinct from that of functional effector or memory T cells. T cell exhaustion generally prevents optimal control of infection and tumors. See, e.g., Wherry E J, Nat Immunol. (2011) 12: 492-499, for additional information about T cell exhaustion. Typically, T cell exhaustion results from the binding of an immune checkpoint protein to at least one of its ligands (e.g. PD1-1 and one of its ligands PD-L1 or PD-L2).

In some embodiments, the subject suffers from a cancer and the method of the present invention is thus suitable for enhancing the proliferation, migration, persistence and/or cytoxic activity of tumor infiltrating cytotoxic T lymphocytes.

As used herein, the term “tumor infiltrating cytotoxic T lymphocyte” refers to the pool of cytotoxic T lymphocytes of the patient that have left the blood stream and have migrated into a tumor.

In particular, the method of the present invention is suitable for the treatment of a cancer characterized by a high tumor infiltration of cytotoxic T lymphocytes that express an immune checkpoint protein. Typically said tumor-infiltration of cytotoxic T lymphocytes is determined by any conventional method in the art. For example, said determination comprises quantifying the density of cytotoxic T lymphocytes that express at least one immune checkpoint protein (e.g. PD-1) in a tumor sample obtained from the patient.

As used herein, the term “tumor tissue sample” means any tissue tumor sample derived from the patient. Said tissue sample is obtained for the purpose of the in vitro evaluation. In some embodiments, the tumor sample may result from the tumor resected from the patient. In some embodiments, the tumor sample may result from a biopsy performed in the primary tumor of the patient or performed in metastatic sample distant from the primary tumor of the patient, for example an endoscopical biopsy performed in the bowel of the patient affected by a colorectal cancer. In some embodiments, the tumor tissue sample encompasses (i) a global primary tumor (as a whole), (ii) a tissue sample from the center of the tumor, (iii) a tissue sample from the tissue directly surrounding the tumor which tissue may be more specifically named the “invasive margin” of the tumor, (iv) lymphoid islets in close proximity with the tumor, (v) the lymph nodes located at the closest proximity of the tumor, (vi) a tumor tissue sample collected prior surgery (for follow-up of patients after treatment for example), and (vii) a distant metastasis. As used herein the “invasive margin” has its general meaning in the art and refers to the cellular environment surrounding the tumor. In some embodiments, the tumor tissue sample, irrespective of whether it is derived from the center of the tumor, from the invasive margin of the tumor, or from the closest lymph nodes, encompasses pieces or slices of tissue that have been removed from the tumor center of from the invasive margin surrounding the tumor, including following a surgical tumor resection or following the collection of a tissue sample for biopsy, for further quantification of one or several biological markers, notably through histology or immunohistochemistry methods, through flow cytometry methods and through methods of gene or protein expression analysis, including genomic and proteomic analysis. The tumor tissue sample can, of course, be patiented to a variety of well-known post-collection preparative and storage techniques (e.g., fixation, storage, freezing, etc.). The sample can be fresh, frozen, fixed (e.g., formalin fixed), or embedded (e.g., paraffin embedded). The tumor tissue sample can be used in microarrays, called as tissue microarrays (TMAs). TMA consists of paraffin blocks in which up to 1000 separate tissue cores are assembled in array fashion to allow multiplex histological analysis. This technology allows rapid visualization of molecular targets in tissue specimens at a time, either at the DNA, RNA or protein level. TMA technology is described in WO2004000992, U.S. Pat. No. 8,068,988, Olli et al 2001 Human Molecular Genetics, Tzankov et al 2005, Elsevier; Kononen et al 1198; Nature Medicine.

In some embodiments, the quantification of density of cytotoxic T lymphocytes that express at least one immune checkpoint protein is determined by immunohistochemistry (IHC). For example, the quantification of the density of cytotoxic T lymphocytes is performed by contacting the tissue tumor tissue sample with a binding partner (e.g. an antibody) specific for a cell surface marker of said cells. Typically, the quantification of density of cytotoxic T lymphocytes is performed by contacting the tissue tumor tissue sample with a set of binding partners (e.g. an antibody) specific for CD8 and for the immune checkpoint protein (e.g. PD-1).

Typically, the density of cytotoxic T lymphocytes that express at least one immune checkpoint protein (e.g. PD-1) is expressed as the number of these cells that are counted per one unit of surface area of tissue sample, e.g. as the number of cells that are counted per cm² or mm² of surface area of tumor tissue sample. In some embodiments, the density of cells may also be expressed as the number of cells per one volume unit of sample, e.g. as the number of cells per cm³ of tumor tissue sample. In some embodiments, the density of cells may also consist of the percentage of the specific cells per total cells (set at 100%).

Immunohistochemistry typically includes the following steps i) fixing the tumor tissue sample with formalin, ii) embedding said tumor tissue sample in paraffin, iii) cutting said tumor tissue sample into sections for staining, iv) incubating said sections with the binding partner specific for the marker, v) rinsing said sections, vi) incubating said section with a secondary antibody typically biotinylated and vii) revealing the antigen-antibody complex typically with avidin-biotin-peroxidase complex. Accordingly, the tumor tissue sample is firstly incubated the binding partners. After washing, the labeled antibodies that are bound to a marker of interest are revealed by the appropriate technique, depending of the kind of label being borne by the labeled antibody, e.g. radioactive, fluorescent or enzyme label. Multiple labelling can be performed simultaneously. Alternatively, the method of the present invention may use a secondary antibody coupled to an amplification system (to intensify staining signal) and enzymatic molecules. Such coupled secondary antibodies are commercially available, e.g. from Dako, EnVision system. Counterstaining may be used, e.g. H&E, DAPI, Hoechst. Other staining methods may be accomplished using any suitable method or system as would be apparent to one of skill in the art, including automated, semi-automated or manual systems. For example, one or more labels can be attached to the antibody, thereby permitting detection of the target protein (i.e the marker). Exemplary labels include radioactive isotopes, fluorophores, ligands, chemiluminescent agents, enzymes, and combinations thereof. In some embodiments, the label is a quantum dot. Non-limiting examples of labels that can be conjugated to primary and/or secondary affinity ligands include fluorescent dyes or metals (e.g. fluorescein, rhodamine, phycoerythrin, fluorescamine), chromophoric dyes (e.g. rhodopsin), chemiluminescent compounds (e.g. luminal, imidazole) and bioluminescent proteins (e.g. luciferin, luciferase), haptens (e.g. biotin). A variety of other useful fluorescers and chromophores are described in Stryer L (1968) Science 162:526-533 and Brand L and Gohlke J R (1972) Annu. Rev. Biochem. 41:843-868. Affinity ligands can also be labeled with enzymes (e.g. horseradish peroxidase, alkaline phosphatase, beta-lactamase), radioisotopes (e.g. 3H, 14C, 32P, 35S or 1251) and particles (e.g. gold). The different types of labels can be conjugated to an affinity ligand using various chemistries, e.g. the amine reaction or the thiol reaction. However, other reactive groups than amines and thiols can be used, e.g. aldehydes, carboxylic acids and glutamine. Various enzymatic staining methods are known in the art for detecting a protein of interest. For example, enzymatic interactions can be visualized using different enzymes such as peroxidase, alkaline phosphatase, or different chromogens such as DAB, AEC or Fast Red. In other examples, the antibody can be conjugated to peptides or proteins that can be detected via a labeled binding partner or antibody. In an indirect IHC assay, a secondary antibody or second binding partner is necessary to detect the binding of the first binding partner, as it is not labeled. The resulting stained specimens are each imaged using a system for viewing the detectable signal and acquiring an image, such as a digital image of the staining. Methods for image acquisition are well known to one of skill in the art. For example, once the sample has been stained, any optical or non-optical imaging device can be used to detect the stain or biomarker label, such as, for example, upright or inverted optical microscopes, scanning confocal microscopes, cameras, scanning or tunneling electron microscopes, canning probe microscopes and imaging infrared detectors. In some examples, the image can be captured digitally. The obtained images can then be used for quantitatively or semi-quantitatively determining the amount of the marker in the sample. Various automated sample processing, scanning and analysis systems suitable for use with immunohistochemistry are available in the art. Such systems can include automated staining and microscopic scanning, computerized image analysis, serial section comparison (to control for variation in the orientation and size of a sample), digital report generation, and archiving and tracking of samples (such as slides on which tissue sections are placed). Cellular imaging systems are commercially available that combine conventional light microscopes with digital image processing systems to perform quantitative analysis on cells and tissues, including immunostained samples. See, e.g., the CAS-200 system (Becton, Dickinson & Co.). In particular, detection can be made manually or by image processing techniques involving computer processors and software. Using such software, for example, the images can be configured, calibrated, standardized and/or validated based on factors including, for example, stain quality or stain intensity, using procedures known to one of skill in the art (see e.g., published U.S. Patent Publication No. US20100136549). The image can be quantitatively or semi-quantitatively analyzed and scored based on staining intensity of the sample. Quantitative or semi-quantitative histochemistry refers to method of scanning and scoring samples that have undergone histochemistry, to identify and quantitate the presence of the specified biomarker (i.e. the marker). Quantitative or semi-quantitative methods can employ imaging software to detect staining densities or amount of staining or methods of detecting staining by the human eye, where a trained operator ranks results numerically. For example, images can be quantitatively analyzed using a pixel count algorithms (e.g., Aperio Spectrum Software, Automated QUantitatative Analysis platform (AQUA® platform), and other standard methods that measure or quantitate or semi-quantitate the degree of staining; see e.g., U.S. Pat. Nos. 8,023,714; 7,257,268; 7,219,016; 7,646,905; published U.S. Patent Publication No. US20100136549 and 20110111435; Camp et al. (2002) Nature Medicine, 8:1323-1327; Bacus et al. (1997) Analyt Quant Cytol Histol, 19:316-328). A ratio of strong positive stain (such as brown stain) to the sum of total stained area can be calculated and scored. The amount of the detected biomarker (i.e. the marker) is quantified and given as a percentage of positive pixels and/or a score. For example, the amount can be quantified as a percentage of positive pixels. In some examples, the amount is quantified as the percentage of area stained, e.g., the percentage of positive pixels. For example, a sample can have at least or about at least or about 0, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 40%, 45%, 50%, 55%, 60%, 65%, 70%, 75%, 80%, 85%, 90%, 95% or more positive pixels as compared to the total staining area. In some embodiments, a score is given to the sample that is a numerical representation of the intensity or amount of the histochemical staining of the sample, and represents the amount of target biomarker (e.g., the marker) present in the sample. Optical density or percentage area values can be given a scaled score, for example on an integer scale. Thus, in some embodiments, the method of the present invention comprises the steps consisting in i) providing one or more immunostained slices of tissue section obtained by an automated slide-staining system by using a binding partner capable of selectively interacting with the marker (e.g. an antibody as above described), ii) proceeding to digitalisation of the slides of step a. by high resolution scan capture, iii) detecting the slice of tissue section on the digital picture iv) providing a size reference grid with uniformly distributed units having a same surface, said grid being adapted to the size of the tissue section to be analyzed, and v) detecting, quantifying and measuring intensity of stained cells in each unit whereby the number or the density of cells stained of each unit is assessed.

In a particular embodiment, quantification of the percentage of cytotoxic T lymphocytes that express at least one immune checkpoint protein (e.g. PD-1) is determined by an automatized microscope which allows measurement of morphometric and fluorescence characteristics in the different cell compartments (membrane/cytoplasm/nuclei) and quantifying preciously the percent of interest cells. Briefly the quantification of percent of cytotoxic T lymphocytes that expression at least one immune checkpoint protein (e.g. PD-1) is performed by following steps: i) providing tissue microarray (TMA) containing RCC samples, ii) TMA samples are stained with anti-CD3, anti-CD8, and anti-PD-1 antibodies, iii) the samples are further stained with an epithelial cell marker to assist in automated segmentation of tumour and stroma, iv) TMA slides are then scanned using a multispectral imaging system, v) the scanned images are processed using an automated image analysis software (e.g.Perkin Elmer Technology) which allows the detection and segmentation of specific tissues through powerful pattern recognition algorithms, a machine-learning algorithm is trained to segment tumor from stroma and identify cells labelled; vi) the percent of cytotoxic T lymphocytes that expression at least one immune checkpoint protein (e.g. PD-1) within the tumour areas is calculated; vii) a pathologist rates lymphocytes percentage; and vii) manual and automated scoring are compared with survival time of the subject.

In some embodiments, the cell density of cytotoxic T lymphocytes is determined in the whole tumor tissue sample, is determined in the invasive margin or centre of the tumor tissue sample or is determined both in the centre and the invasive margin of the tumor tissue sample.

Accordingly a further object of the present invention relates to a method of treating cancer in a patient in need thereof comprising i) quantifying the density of cytotoxic T lymphocytes that express at least one immune checkpoint protein (e.g. PD-1) in a tumor tissue sample obtained from the patient ii) comparing the density quantified at step i) with a predetermined reference value and iii) administering to the patient a therapeutically effective amount of Sulconazole when the density quantified at step i) is higher than the predetermined reference value.

As used herein, the term “the predetermined reference value” refers to a threshold value or a cut-off value. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. For example, retrospective measurement of cell densities in properly banked historical subject samples may be used in establishing the predetermined reference value. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. For example, after quantifying the cell density in a group of reference, one can use algorithmic analysis for the statistic treatment of the measured densities in samples to be tested, and thus obtain a classification standard having significance for sample classification. ROC curve is mainly used for clinical biochemical diagnostic tests. ROC curve is a comprehensive indicator that reflects the continuous variables of true positive rate (sensitivity) and false positive rate (1-specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of diagnosis. On the ROC curve, the point closest to the far upper left of the coordinate diagram is a critical point having both high sensitivity and high specificity values. The AUC value of the ROC curve is between 1.0 and 0.5. When AUC>0.5, the diagnostic result gets better and better as AUC approaches 1. When AUC is between 0.5 and 0.7, the accuracy is low. When AUC is between 0.7 and 0.9, the accuracy is moderate. When AUC is higher than 0.9, the accuracy is quite high. This algorithmic method is preferably done with a computer. Existing software or systems in the art may be used for the drawing of the ROC curve, such as: MedCalc 9.2.0.1 medical statistical software, SPSS 9.0, ROCPOWER.SAS, DESIGNROC.FOR, MULTIREADER POWER.SAS, CREATE-ROC.SAS, GB STAT VI0.0 (Dynamic Microsystems, Inc. Silver Spring, Md., USA), etc.

In some embodiments, Sulconazole is administered to the subject in combination with at least one immune checkpoint inhibitor. Examples of immune checkpoint inhibitor includes PD-1 antagonists, PD-L1 antagonists, PD-L2 antagonists, CTLA-4 antagonists, VISTA antagonists, TIM-3 antagonists, LAG-3 antagonists, IDO antagonists, KIR2D antagonists, A2AR antagonists, B7-H3 antagonists, B7-H4 antagonists, and BTLA antagonists.

In some embodiments, PD-1 (Programmed Death-1) axis antagonists include PD-1 antagonist (for example anti-PD-1 antibody), PD-L1 (Programmed Death Ligand-1) antagonist (for example anti-PD-L1 antibody) and PD-L2 (Programmed Death Ligand-2) antagonist (for example anti-PD-L2 antibody). In some embodiments, the anti-PD-1 antibody is selected from the group consisting of MDX-1106 (also known as Nivolumab, MDX-1106-04, ONO-4538, BMS-936558, and Opdivo®), Merck 3475 (also known as Pembrolizumab, MK-3475, Lambrolizumab, Keytruda®, and SCH-900475), and CT-011 (also known as Pidilizumab, hBAT, and hBAT-1). In some embodiments, the PD-1 binding antagonist is AMP-224 (also known as B7-DCIg). In some embodiments, the anti-PD-L1 antibody is selected from the group consisting of YW243.55.570, MPDL3280A, MDX-1105, and MEDI4736. MDX-1105, also known as BMS-936559, is an anti-PD-L1 antibody described in WO2007/005874. Antibody YW243.55. S70 is an anti-PD-L1 described in WO 2010/077634 A1. MEDI4736 is an anti-PD-L1 antibody described in WO2011/066389 and US2013/034559. MDX-1106, also known as MDX-1106-04, ONO-4538 or BMS-936558, is an anti-PD-1 antibody described in U.S. Pat. No. 8,008,449 and WO2006/121168. Merck 3745, also known as MK-3475 or SCH-900475, is an anti-PD-1 antibody described in U.S. Pat. No. 8,345,509 and WO2009/114335. CT-011 (Pidizilumab), also known as hBAT or hBAT-1, is an anti-PD-1 antibody described in WO2009/101611. AMP-224, also known as B7-DCIg, is a PD-L2-Fc fusion soluble receptor described in WO2010/027827 and WO2011/066342. Atezolimumab is an anti-PD-L1 antibody described in U.S. Pat. No. 8,217,149. Avelumab is an anti-PD-L1 antibody described in US 20140341917. CA-170 is a PD-1 antagonist described in WO2015033301 & WO2015033299. Other anti-PD-1 antibodies are disclosed in U.S. Pat. No. 8,609,089, US 2010028330, and/or US 20120114649. In some embodiments, the PD-1 inhibitor is an anti-PD-1 antibody chosen from Nivolumab, Pembrolizumab or Pidilizumab. In some embodiments, PD-L1 antagonist is selected from the group comprising of Avelumab, BMS-936559, CA-170, Durvalumab, MCLA-145, SP142, STI-A1011, STIA1012, STI-A1010, STI-A1014, A110, KY1003 and Atezolimumab and the preferred one is Avelumab, Durvalumab or Atezolimumab.

In some embodiments, CTLA-4 (Cytotoxic T-Lymphocyte Antigen-4) antagonists are selected from the group consisting of anti-CTLA-4 antibodies, human anti-CTLA-4 antibodies, mouse anti-CTLA-4 antibodies, mammalian anti-CTLA-4 antibodies, humanized anti-CTLA-4 antibodies, monoclonal anti-CTLA-4 antibodies, polyclonal anti-CTLA-4 antibodies, chimeric anti-CTLA-4 antibodies, MDX-010 (Ipilimumab), Tremelimumab, anti-CD28 antibodies, anti-CTLA-4 adnectins, anti-CTLA-4 domain antibodies, single chain anti-CTLA-4 fragments, heavy chain anti-CTLA-4 fragments, light chain anti-CTLA-4 fragments, inhibitors of CTLA-4 that agonize the co-stimulatory pathway, the antibodies disclosed in PCT Publication No. WO 2001/014424, the antibodies disclosed in PCT Publication No. WO 2004/035607, the antibodies disclosed in U.S. Publication No. 2005/0201994, and the antibodies disclosed in granted European Patent No. EP 1212422 B. Additional CTLA-4 antibodies are described in U.S. Pat. Nos. 5,811,097; 5,855,887; 6,051,227; and 6,984,720; in PCT Publication Nos. WO 01/14424 and WO 00/37504; and in U.S. Publication Nos. 2002/0039581 and 2002/086014. Other anti-CTLA-4 antibodies that can be used in a method of the present invention include, for example, those disclosed in: WO 98/42752; U.S. Pat. Nos. 6,682,736 and 6,207,156; Hurwitz et al., Proc. Natl. Acad. Sci. USA, 95(17): 10067-10071 (1998); Camacho et al., J. Clin: Oncology, 22(145): Abstract No. 2505 (2004) (antibody CP-675206); Mokyr et al., Cancer Res., 58:5301-5304 (1998), and U.S. Pat. Nos. 5,977,318, 6,682,736, 7,109,003, and 7,132,281. A preferred clinical CTLA-4 antibody is human monoclonal antibody (also referred to as MDX-010 and Ipilimumab with CAS No. 477202-00-9 and available from Medarex, Inc., Bloomsbury, N.J.) is disclosed in WO 01/14424. With regard to CTLA-4 antagonist (antibodies), these are known and include Tremelimumab (CP-675,206) and Ipilimumab.

Other immune-checkpoint inhibitors include lymphocyte activation gene-3 (LAG-3) inhibitors, such as IMP321, a soluble Ig fusion protein (Brignone et al., 2007, J. Immunol. 179:4202-4211). Other immune-checkpoint inhibitors include B7 inhibitors, such as B7-H3 and B7-H4 inhibitors. In particular, the anti-B7-H3 antibody MGA271 (Loo et al., 2012, Clin. Cancer Res. July 15 (18) 3834). Also included are TIM-3 (T-cell immunoglobulin domain and mucin domain 3) inhibitors (Fourcade et al., 2010, J. Exp. Med. 207:2175-86 and Sakuishi et al., 2010, J. Exp. Med. 207:2187-94). As used herein, the term “TIM-3” has its general meaning in the art and refers to T cell immunoglobulin and mucin domain-containing molecule 3. The natural ligand of TIM-3 is galectin 9 (Gal9). Accordingly, the term “TIM-3 inhibitor” as used herein refers to a compound, substance or composition that can inhibit the function of TIM-3. For example, the inhibitor can inhibit the expression or activity of TIM-3, modulate or block the TIM-3 signalling pathway and/or block the binding of TIM-3 to galectin-9. Antibodies having specificity for TIM-3 are well known in the art and typically those described in WO2011155607, WO2013006490 and WO2010117057.

In some embodiments, the immune checkpoint inhibitor is an IDO inhibitor. Examples of IDO inhibitors are described in WO 2014150677. Examples of IDO inhibitors include without limitation 1-methyl-tryptophan (IMT), β-(3-benzofuranyl)-alanine, β-(3-benzo(b)thienyl)-alanine), 6-nitro-tryptophan, 6-fluoro-tryptophan, 4-methyl-tryptophan, 5-methyl tryptophan, 6-methyl-tryptophan, 5-methoxy-tryptophan, 5-hydroxy-tryptophan, indole 3-carbinol, 3,3′-diindolylmethane, epigallocatechin gallate, 5-Br-4-C1-indoxyl 1,3-diacetate, 9-vinylcarbazole, acemetacin, 5-bromo-tryptophan, 5-bromoindoxyl diacetate, 3-Amino-naphtoic acid, pyrrolidine dithiocarbamate, 4-phenylimidazole a brassinin derivative, a thiohydantoin derivative, a β-carboline derivative or a brassilexin derivative. Preferably the IDO inhibitor is selected from 1-methyl-tryptophan, β-(3-benzofuranyl)-alanine, 6-nitro-L-tryptophan, 3-Amino-naphtoic acid and β-[3-benzo(b)thienyl]-alanine or a derivative or prodrug thereof.

According to the invention, Sulconazole is administered to the patient in a therapeutically effective amount. By a “therapeutically effective amount” is meant a sufficient amount of the active ingredient for treating or reducing the symptoms at reasonable benefit/risk ratio applicable to any medical treatment. It will be understood that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination with the active ingredients; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, typically from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

Typically the active ingredient of the present invention (e.g. proprotein convertase inhibitor) is combined with pharmaceutically acceptable excipients, and optionally sustained-release matrices, such as biodegradable polymers, to form pharmaceutical compositions. The term “Pharmaceutical” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. In the pharmaceutical compositions of the present invention, the active ingredients of the invention can be administered in a unit administration form, as a mixture with conventional pharmaceutical supports. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

A further object of the present invention relates to an in vitro or ex vivo method of reducing the expression of at least one immune checkpoint protein in a population of immune cells comprising contacting the population of T cells with an amount of Sulconazole.

In some embodiments, the method is particularly suitable for reducing the expression of at least one immune checkpoint protein in a population of macrophages, monocytes or dendritic cells.

In some embodiments, the method is particularly suitable for reducing the expression of at least immune checkpoint protein in a population of immune effector cells. Preferred effector cells include, but are not limited to T cells, natural killer (NK) cells, and natural killer T (NKT) cells.

As used herein, the term “T cells” has its general meaning in the art and represent an important component of the immune system that plays a central role in cell-mediated immunity. T cells are known as conventional lymphocytes as they recognize the antigen with their TCR (T cell receptor for the antigen) with presentation or restriction by molecules of the complex major histocompatibility. There are several subsets of T cells each having a distinct function such as CD8+ T cells, CD4+ T cells, Gamma delta T cells, and Tregs.

In some embodiments, the population of T cells is a population of cytotoxic T lymphocytes (as defined above). Naive CD8+ T cells have numerous acknowledged biomarkers known in the art. These include CD45RA+CCR7+HLA−DR−CD8+ and the TCR chain is formed of alpha chain (α) and beta chain (β). Persisting (central memory and effector memory), non-persisting (effector or exhausted subpopulations), anergic/tolerant and senescent regulatory CD8+ T cells can be discriminated on their differential expression of surface markers including (but not limited to) CCR7, CD44, CD62L, CD122; CD127; IL15R, KLRG1, CD57, CD137, CD45RO, CD95, PD-1 CTLA, Lag3 and transcription factors such as T-bet/Eomes, BCL6, Blimp-1, STAT3/4/5 ID2/3, NFAT, FoxP3.

In some embodiments, the population of T cells is a population of CD4+ T cells. The term “CD4+ T cells” (also called T helper cells or TH cells) refers to T cells which express the CD4 glycoprotein on their surfaces and which assist other white blood cells in immunologic processes, including maturation of B cells into plasma cells and memory B cells, and activation of cytotoxic T cells and macrophages. CD4+ T cells become activated when they are presented with peptide antigens by MHC class II molecules, which are expressed on the surface of antigen-presenting cells (APCs). Once activated, they divide rapidly and secrete cytokines that regulate or assist in the active immune response. These cells can differentiate into one of several subtypes, including TH1, TH2, TH3, TH17, TH9, TFH or Treg, which secrete different cytokines to facilitate different types of immune responses. Signaling from the APC directs T cells into particular subtypes. In addition to CD4, the TH cell surface biomarkers known in the art include CXCR3 (Th1), CCR4, Crth2 (Th2), CCR6 (Th17), CXCRS (Tfh) and as well as subtype-specific expression of cytokines and transcription factors including T-bet, GATA3, EOMES, RORyT, BCL6 and FoxP3.

In some embodiments, the population of T cells is a population of gamma delta T cells. Gamma delta T cells normally account for 1 to 5% of peripheral blood lymphocytes in a healthy individual (human, monkey). They are involved in mounting a protective immune response, and it has been shown that they recognize their antigenic ligands by a direct interaction with antigen, without any presentation by MHC molecules of antigen-presenting cells. Gamma 9 delta 2 T cells (sometimes also called gamma 2 delta 2 T cells) are gamma delta T cells bearing TCR receptors with the variable domains Vy9 and V62. They form the majority of gamma delta T cells in human blood. When activated, gamma delta T cells exert potent, non-MHC restricted cytotoxic activity, especially efficient at killing various types of cells, particularly pathogenic cells. These may be cells infected by a virus (Poccia et al., J. Leukocyte Biology, 1997, 62: 1-5) or by other intracellular parasites, such as mycobacteria (Constant et al., Infection and Immunity, December 1995, vol. 63, no. 12: 4628-4633) or protozoa (Behr et al., Infection and Immunity, 1996, vol. 64, no. 8: 2892-2896). They may also be cancer cells (Poccia et al., J. Immunol., 159: 6009-6015; Fournie and Bonneville, Res. Immunol., 66th Forum in Immunology, 147: 338-347). The possibility of modulating the activity of said cells in vitro, ex vivo or in vivo would therefore provide novel, effective therapeutic approaches in the treatment of various pathologies such as infectious diseases (particularly viral or parasitic), cancers, allergies, and even autoimmune and/or inflammatory disorders.

In some embodiments, the population of T cells is a population of CAR-T cells. As used herein the term “CAR-T cell” refers to a T lymphocyte that has been genetically engineered to express a CAR. The definition of CAR T-cells encompasses all classes and subclasses of T-lymphocytes including CD4+, CD8+ T cells, gamma delta T cells as well as effector T cells, memory T cells, regulatory T cells, and the like. The T lymphocytes that are genetically modified may be “derived” or “obtained” from the subject who will receive the treatment using the genetically modified T cells or they may “derived” or “obtained” from a different subject. The term “chimeric antigen receptors (CARs),” as used herein, may refer to artificial T-cell receptors T-bodies, single-chain immunoreceptors, chimeric T-cell receptors, or chimeric immunoreceptors, for example, and encompass engineered receptors that graft an artificial specificity onto a particular immune effector cell. CARs may be employed to impart the specificity of a monoclonal antibody onto a T cell, thereby allowing a large number of specific T cells to be generated, for example, for use in adoptive cell therapy. In some embodiments, CARs comprise an intracellular activation domain, a transmembrane domain, and an extracellular domain that may vary in length and comprises a tumor associated antigen binding region. In particular aspects, CARs comprise fusions of single-chain variable fragments (scFv) derived from monoclonal antibodies, fused to CD3-zeta a transmembrane domain and endodomain. In some embodiments, CARs comprise domains for additional co-stimulatory signaling, such as CD3-zeta, FcR, CD27, CD28, CD137, DAP10, and/or OX40. In some embodiments, molecules can be co-expressed with the CAR, including co-stimulatory molecules, reporter genes for imaging (e.g., for positron emission tomography), gene products that conditionally ablate the T cells upon addition of a pro-drug, homing receptors, chemokines, chemokine receptors, cytokines, and cytokine receptors.

In some embodiments, the population of T cells is specific for an antigen. The term “antigen” (“Ag”) as used herein refers to protein, peptide, nucleic acid or tissue or cell preparations capable of eliciting a T cell response. In some embodiments, the antigen is a tumor-associated antigen (TAA). Examples of TAAs include, without limitation, melanoma-associated Ags (Melan-A/MART-1, MAGE-1, MAGE-3, TRP-2, melanosomal membrane glycoprotein gp100, gp75 and MUC-1 (mucin-1) associated with melanoma); CEA (carcinoembryonic antigen) which can be associated, e.g., with ovarian, melanoma or colon cancers; folate receptor alpha expressed by ovarian carcinoma; free human chorionic gonadotropin beta (hCGP) subunit expressed by many different tumors, including but not limited to ovarian tumors, testicular tumors and myeloma; HER-2/neu associated with breast cancer; encephalomyelitis antigen HuD associated with small-cell lung cancer; tyrosine hydroxylase associated with neuroblastoma; prostate-specific antigen (PSA) associated with prostate cancer; CA125 associated with ovarian cancer; and the idiotypic determinants of a B-cell lymphoma that can generate tumor-specific immunity (attributed to idiotype-specific humoral immune response), Mesothelin associated with pancreatic, ovarian and lung cancer, P53 associated with ovarian, colorectal, non small cell lung cancer, NY-ESO-1 associated with testis, ovarian cancer, EphA2 associated with breast, prostate, lung cancer, EphA3 associated with colorectal carcinoma, Survivin associated with lung, breast, pancreatic, ovarian cancer, HPV E6 and E7 associated with cervical cancer, EGFR associated with NSCL cancer. Moreover, Ags of human T cell leukemia virus type 1 have been shown to induce specific cytotoxic T cell responses and anti-tumor immunity against the virus-induced human adult T-cell leukemia (ATL). Other leukemia Ags can equally be used. Tumor-associated antigens which can be used in the present invention are disclosed in the book “Categories of Tumor Antigens” (Hassane M. et al Holland-Frei Cancer Medicine (2003). 6th edition) and the review Gregory T. et al (“Novel cancer antigens for personalized immunotherapies: latest evidence and clinical potential” Ther Adv Med Oncol. 2016; 8(1): 4-31) all of which are herein incorporated by reference. In some embodiments, the tumor-associated antigen is melanoma-associated Ags.

Typically, the population of T cells is prepared from a PBMC. The term “PBMC” or “peripheral blood mononuclear cells” or “unfractionated PBMC”, as used herein, refers to whole PBMC, i.e. to a population of white blood cells having a round nucleus, which has not been enriched for a given sub-population. Cord blood mononuclear cells are further included in this definition. Typically, the PBMC sample according to the invention has not been subjected to a selection step to contain only adherent PBMC (which consist essentially of >90% monocytes) or non-adherent PBMC (which contain T cells, B cells, natural killer (NK) cells, NK T cells and DC precursors). A PBMC sample according to the invention therefore contains lymphocytes (B cells, T cells, NK cells, NKT cells), monocytes, and precursors thereof. Typically, these cells can be extracted from whole blood using Ficoll, a hydrophilic polysaccharide that separates layers of blood, with the PBMC forming a cell ring under a layer of plasma. Additionally, PBMC can be extracted from whole blood using a hypotonic lysis buffer, which will preferentially lyse red blood cells. Such procedures are known by a skilled person in the art. For example, the initial cell preparation consists of PBMCs from fresh or frozen (cytopheresed) blood. Isolated T cell (or APC) can be analysed in flux cytometry. Several doses of the T cells (or APC) cellular product can be manufactured from one frozen cytopheresis. Typically, 100 million frozen PBMCs from cytopheresis yield 1 to 5 billion cells with the classical method of preparation. Standard methods for purifying and isolating T cells are well known in the art. For instance, cell sorting is a current protocol that may be used to isolate and purify the obtained CTLs. Typically, multimers (e.g. tetramers or pentamers) consisting of MHC class 1 molecules loaded with the immunogenic peptide are used. To produce multimers, the carboxyl terminus of an MHC molecule, such as, for example, the HLA A2 heavy chain, is associated with a specific peptide epitope, and treated so as to form a multimer complex having bound hereto a suitable reporter molecule, preferably a fluorochrome such as, for example, fluoroscein isothiocyanate (FITC), phycoerythrin, phycocyanin or allophycocyanin. The multimers produced bind to the distinct set of CD8+ T cell receptors (TcRs) on a subset of CD8+ T cells to which the peptide is MHC class I restricted. Following binding, and washing of the T cells to remove unbound or non-specifically bound multimer, the number of CD8+ cells binding specifically to the HLA-peptide multimer may be quantified by standard flow cytometry methods, such as, for example, using a FACS Calibur Flow cytometer (Becton Dickinson). The multimers can also be attached to paramagnetic particles or magnetic beads to facilitate removal of non-specifically bound reporter and cell sorting. Such particles are readily available from commercial sources (eg. Beckman Coulter, Inc., San Diego, Calif., USA).

In some embodiments, once the selected naive T cells (e.g. naive CD8+ T cells) are purified they are subsequently admixed and incubated the population of antigen presenting cells (APCs) for a time sufficient to activate and enrich for a desired population of activated T cells, such as activated helper T cells, and preferably, CTLs or CD8+ memory T cells. Such activated T cells preferably are activated in a peptide-specific manner. The ratio of substantially separated naive T cells to APCs may be optimized for the particular individual, e.g., in light of individual characteristics such as the amenability of the individual's lymphocytes to culturing conditions and the nature and severity of the disease or other condition being treated. Any culture medium suitable for growth, survival and differentiation of T cells is used for the coculturing step. Typically, the base medium can be RPMI 1640, DMEM, IMDM, X-VIVO or AIM-V medium, all of which are commercially available standard media. Typically, the naive T cells are contacted with the APCs of the present invention for a sufficient time to activate a CTL response. In some embodiments, one or more selected cytokines that promote activated T cell growth, proliferation, and/or differentiation are added to the culture medium. The selection of appropriate cytokines will depend on the desired phenotype of the activated T cells that will ultimately comprise the therapeutic composition or cell therapy product. For instance cytokines include IL-1, IL-2, IL-7, IL-4, IL-5, IL-6, IL-12, IFN-γ, and TNF-α. In some embodiments, the culture medium comprises antibodies. Exemplary antibodies include monoclonal anti-CD3 antibodies, such as that marked as ORTHOCLONE OKT®3 (muromonab-CD3).

In some embodiments, the population of T cells is contacted with Sulconazole for a time sufficient for to reduce the expression of checkpoint proteins. For instance, the population of T cells and Sulconazole are contacted with each other for 1, 2, 3, 4, 5, 6, 7, 8, 9, 10, 11, 12, 13, 14, 15, 16, 17, 18, 19, 20, 21, 22, 23, 24, 25, 26, 27, 28 or 30 hours. Typically, Sulconazole is added in the culture medium where the population of T cells is cultured. In some embodiments, Sulconazole is added when the population of T cells is activated (for instance in presence of a population of APC).

Once the population of T cells is obtained, functionality of the cells may be evaluated according to any standard method which typically include a cytotoxic assay. Cell surface phenotype of the cells with the appropriate binding partners can also be confirmed. Quantifying the secretion of various cytokines may also be performed. Methods for quantifying secretion of a cytokine in a sample are well known in the art. For example, any immunological method such as but not limited to ELISA, multiplex strategies, ELISPOT, immunochromatography techniques, proteomic methods, Western blotting, FACS, or Radioimmunoassays may be applicable to the present invention.

The population of T cells obtained by the method of the present invention may find various applications. More particularly, the population of T cells is suitable for the adoptive immunotherapy. The in vitro or ex vivo method of the present invention is particularly suitable for preventing T cell exhaustion when the population of T cells is administered to a patient for adoptive immunotherapy. As used herein, the term “adoptive immunotherapy” refers the administration of donor or autologous T lymphocytes for the treatment of a disease or disease condition, wherein the disease or disease condition results in an insufficient or inadequate immune response. Adoptive immunotherapy is an appropriate treatment for any disease or disease condition where the elimination of infected or transformed cells has been demonstrated to be achieved by a specific population of T cells. Exemplary diseases, disorders, or conditions that may be treated with the population of T cells as prepared according to the present invention include, for example, include immune disorders, such as immune deficiency disorders, autoimmune disorders, and disorders involving a compromised, insufficient, or ineffective immune system or immune system response; infections, such as viral infections, bacterial infections, mycoplasma infections, fungal infections, and parasitic infections; and cancers.

The invention will be further illustrated by the following figures and examples. However, these examples and figures should not be interpreted in any way as limiting the scope of the present invention.

### FIGURES

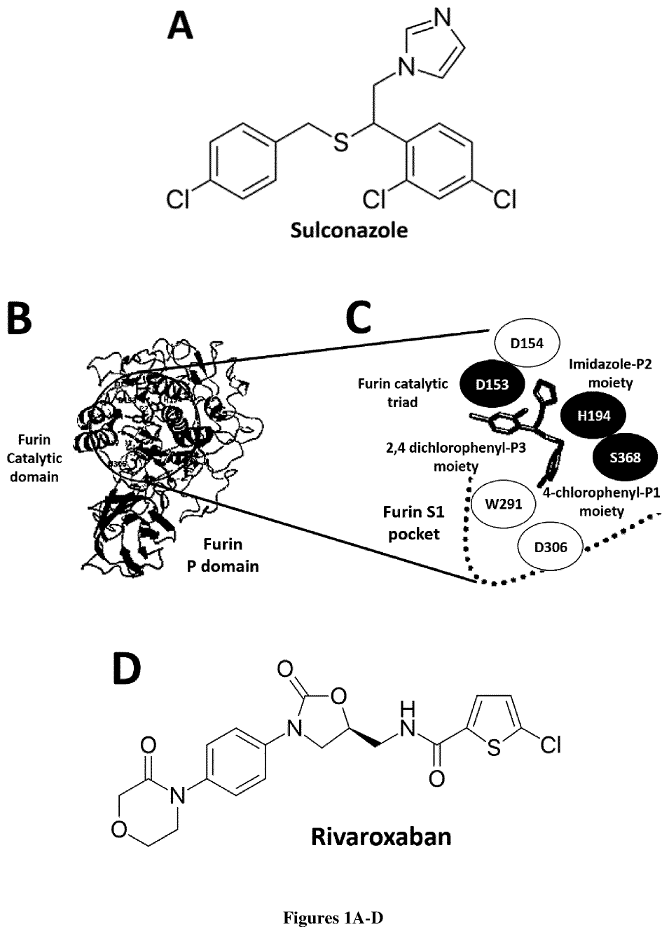

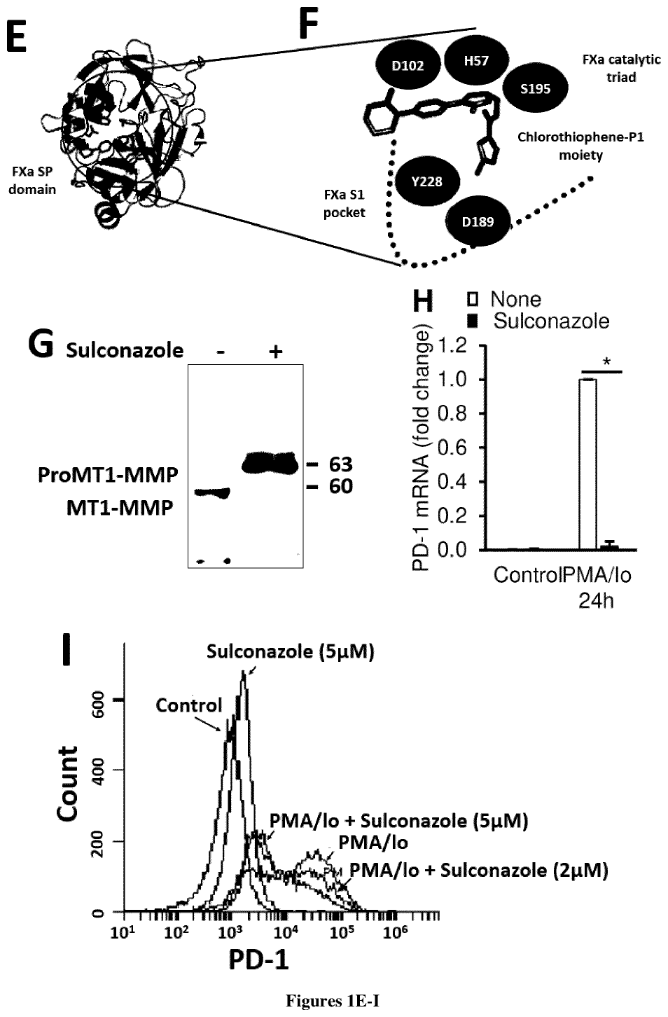

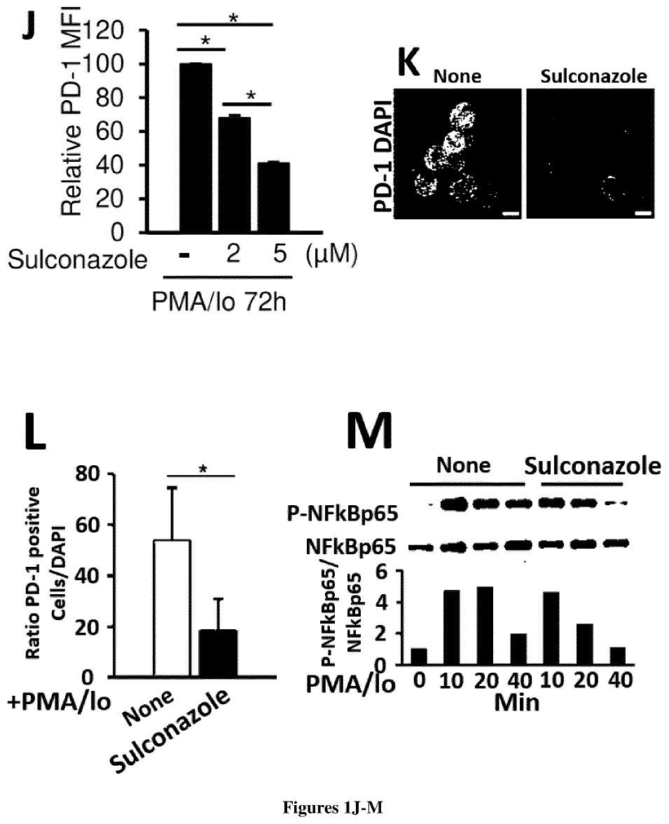

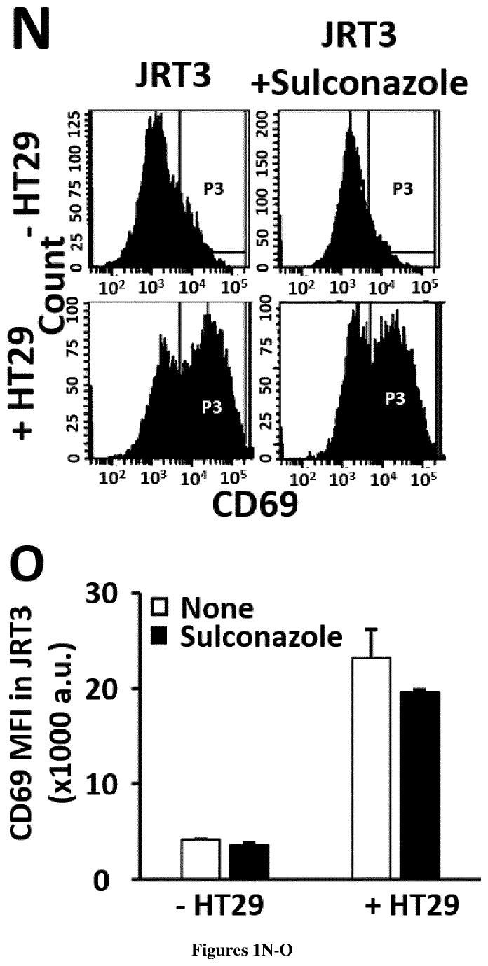

FIG. 1. Proposed docking pose of Sulconazole in the binding site of human furin.
(A) Chemical structure of Sulconazole. (B, C) The crystal structure of human furin is shown with a view down the catalytic site (C). The catalytic triad (S368, H194, D153) and the residue at the bottom of the specificity pocket (D306) are in red (PDB file 4OMC). Two residues (D154 and W291) likely important for the interaction with Sulconazole are shown in magenta. The docking computations suggest that the 4-chlorophenyl-P1 moiety of Sulconazole could bind to the S1 pocket and replace the positively charged benzamidine group seen in the X-ray structure by making interactions with furin aromatic residue W291, similarly to the one observed between the chlorothiophene moiety of Rivaroxaban and factor Xa Y228 (D-F). The imidazole P2 moiety of Sulconazole could have electrostatic interactions with D154. (D) Chemical structure of Rivaroxaban. (E, F) The crystal structure of human coagulation factor Xa serine protease (SP) domain in complex with the approved anticoagulant drug Rivaroxaban is shown to help the analysis of the docking computations carried out on furin (PDB file 2W26). The catalytic triad residues (H57, D102, S195) and the residue at the bottom of the specificity S1 pocket (D189) are in red. An important aromatic residue for the interaction with the P1 group of Rivaroxaban is shown in magenta. The small chemical inhibitor Rivaroxaban is shown in a stick representation. (G) Inhibition of PCs activity in cells by Sulconazole, as demonstrated by the repression of MT1-MMP cleavage and the accumulation of its unprocessed form (ProMT1-MMP) as shown by Western blot analysis. (H) PD-1 mRNA levels following PMA/Io stimulation of T cells, as assessed by Real-time qPCR. Data are represented as fold change to PMA/Io-activated cells assigned 1. (I, J) Flow cytometry histogram and quantification of PD-1 expression following PMA/Io stimulation of T cells treated with indicated concentrations of Sulconazole. (K) Representative confocal microscopy images of PD-1 immunofluorescence from T cells in the absence and presence of Sulcanozole. Scale bar 5 μm. (L) Immunofluorescence image quantification of PD-1 positive cells relative to total cells (DAPI). (M) Western blot and densitometry analysis of phospho-NFκBp65 after treatment of T cells with Sulconazole (2 μM) at indicated time points. (N, O) Flow cytometry histograms (N) and analysis (O) of TCR-activation marker CD69 in JRT3 cells upon binding to HT29 cells in the presence and absence of Sulconazole for 24 h. Data represented as mean±SEM from 3 (H, J) and 2 (0) independent experiments. Data represented as mean±SD from four images and three different areas per image*p<0.05.

### EXAMPLE

Methods

Human PBMCs, T Cells and Tumor Samples

All specimens were obtained following written informed consent approved by Bergonie Institute and Hospital Pellegrin. Patient consent forms for all samples were obtained at the time of tissue acquisition. Biopsies were de-identified. Matched whole blood and freshly resected colon tumor tissues and their non-tumoregenous counterparts were obtained from Bergonie Institute, Bordeaux, France. Fresh whole blood from healthy donors was obtained from Hospital Pellegrin, Bordeaux, France. Samples were processed for RNA and histology studies. Human peripheral blood mononuclear cells (hPBMCs) were isolated from healthy donors and colon cancer patients and were directly used for RNA/protein extraction, or used for CD8+ T cell isolation. Tumor-infiltrating CD8+ T cells were isolated from colon tumor samples freshly harvested following manufacturer's instructions (Miltenyi Biotec). Please refer to Supplementary Information for detailed procedures.

Cell Lines

Human colon carcinoma cell lines HT29 (microsatellite-instable (MSI)) and HCT116 (microsatellite-stable (MSS)) cells, BALB/c syngeneic colon carcinoma CT26 cell line, and human T cell lines Jurkat, J.RT3-T3.5 (JRT3), Myla, SeAx and HUT-78, were cultured in DMEM or RPMI 1640 complete media. The characteristics and the origin of the control (0) and stably α1-PDX-expresssing Jurkat cells (Jurkat-PDX) and CT26 cells (CT26-PDX) were described previously (8, 20). Please refer to Supplementary Information for detailed procedures.

T Cell Activation

Activation of TCR signaling was performed either with phorbol myristate acetate (PMA) and Ionomycin (Io) or with plate-bound anti-CD3. Please refer to Supplementary Information for detailed procedures.

Proliferation Assay

Proliferation of Jurkat-Ø and PDX cells following PMA and Ionomycin activation was determined using a Countess II Automated Cell Counter (Invitrogen). Please refer to Supplementary Information for detailed procedures.

JRT3 Functional Assay

The Jurkat T cell line J.RT3-T3.5 (JRT3) stably expressing the human LES-γδ TCR (JRT3-LES) was incubated with the colon cancer cell line HT29 overexpressing the endothelial protein C receptor (HT29-EPCR) (34). The activation of JRT3-LES cells was evaluated by the expression of CD69. Please refer to Supplementary Information for detailed procedures.

Cytometric Bead Array (CBA)

CBA was used to measure the concentration of cytotoxins released by primary human CD8+ T cell populations. Please refer to Supplementary Information for detailed procedures.

Cytotoxicity Assay

Susceptibility of HT29 and HCT116 to PBMC-mediated cytotoxicity was determined using a carboxyfluorescein diacetate succinimidyl ester (CFSE)-based assay. Please refer to Supplementary Information for detailed procedures.

Flow Cytometry Analysis

Flow cytometry analyzed were performed on Single cell suspension of hPBMCs and T cells using BD Accuri C6 software, or Diva (BD Biosciences) and FlowJo 9.3.2 (TreeStar) softwares (flow cytometry facility of TBM Core). Please refer to Supplementary Information for detailed procedures.

Measurement of PC Activity

The effect of PC inhibitors (CMK, al-PDX expression in cells) on PC activity in cells and tissues was assessed by the evaluation of the enzymes' ability to digest the universal PC substrate, the fluorogenic peptide pERTKR-MCA, as previously described (8). Please refer to Supplementary Information for detailed procedures.

Protein Extraction

For total protein extraction, cells were washed with PBS prior to the addition of RIPA lyses buffer. For nuclear fractionation, cells were lysed using a NE-PER nuclear and cytoplasmic extraction reagent kit (Thermo Scientific) following manufacturer's instructions. Please refer to Supplementary Information for detailed procedures and primary antibodies used.

Cytosolic Free-Calcium Measurement

Jurkat-Ø and PDX were loaded with fura-2 by incubation with 4 μM fura-2/AM (Fura-2 acetoxymethyl ester, Molecular Probes) and 2.5 mM probenecid for 30 min at 37° C. in the dark. Changes in [Ca⁺⁺]c were monitored using the fura-2 340/380 fluorescence ratio and calibrated according to Grynkiewicz et al. (35). Ca⁺⁺ release and entry were estimated using the integral of the rise in [Ca⁺⁺]c for 3 min after the addition of PMA+Ionomycin or CaCl₂), respectively. Please refer to Supplementary Information for detailed procedures.

Search for Novel Furin Inhibitors Via Structure-Based Virtual of Approved Drugs and Experimental Screening

In order to find novel potential inhibitors of furin, we used structure-based virtual screening (9). A compound collection of approved drugs was generated by combining molecules from DrugBank (10), DrugCentral (11), the NCGC Pharmaceutical Collection (12) and SWEETLEAD. The crystal structure of human furin in complex with peptide-like competitive inhibitors (PDB file 4OMC) was used. Please refer to Supplementary Information for detailed procedures.

Statistical Analysis

Statistical details can be found in Results, Figure and Figure Legend sections. Data are shown as mean±S.E.M. or mean±SD. Analysis of statistical significance was performed using Student's t-test or one-way ANOVA followed by Bonferroni's comparison as a post hoc test. Statistical significance was estimated when P<0.05.

Results

In order to repurpose approved drugs against furin, we first generated a hand-curated database of approved drugs (small molecules). About 10,000 compounds were first downloaded from different databases and only molecules that could be docked with a reasonable chance of success were kept (i.e., MW less that 900 Da and a number of rotatable bonds inferior to 20). We obtained a collection of 2082 molecules acting in different therapeutic areas. Of the identified molecules with the highest docking scores (e.g., an estimation of binding affinity), Sulconazole (FIG. 1A), a broad-spectrum anti-fungal agent, was found to be of potential interest. The main role of this drug is expected to be the inhibition of the fungal cytochrome P-450 isoenzyme, C-14-alpha demethylase. Such molecule was however found to be promiscuous in some assay types (4). Yet, this drug and several related synthetized analogs were found to inhibit specifically a protein-protein interaction involving the WW domains of cellular ubiquitin ligases of the Nedd4 family and the PPxY motif of the adenoviral capsid protein VI (5), suggesting that sulconazole could be interesting for repositioning purposes and to probe molecular mechanisms if appropriate control experiments are performed. As illustrated in FIG. 1, a potential binding mode for Sulconazole in the binding site of furin is suggested (FIGS. 1B and 1C). Furin is a member of the PCs of subtilisin-like endoproteinases that cleaves peptide segments displaying a basic residue (eg., arginine) at the P1 position. As Sulconazole does not display a positively charged group at this position, we thought to investigate related enzymes such as to gain additional knowledge over the docked pose. The crystal structure of human coagulation factor Xa serine protease (SP) domain in complex with the approved anticoagulant drug Rivaroxaban was used for this purpose (6). Many serine proteases have substrates or inhibitors with a positively charged P1 residue that makes favorable interactions with the negatively charged D189 at the bottom of the 51 specificity pocket (FIG. 1D-F). Rivaroxaban displays at this position a chlorothiophene moiety that interacts strongly with a Tyr residue (Y228), and as such a highly basic P1 group such as amidine (arginine-P1 mimetics) is not required, enabling high potency and good oral bioavailability in contrast to molecules having a positively charged P1 group. By comparison, the 4-chlorophenyl-P1 moiety of Sulconazole could bind to the 51 pocket and replace the positively charged benzamidine group seen in the X-ray structure of furin complexed with a peptide-like inhibitors (7) by making favorable interactions with the aromatic residue W291 of furin, in a manner similar to the one between FXa and Rivaroxaban. Further, the imidazole P2 moiety of Sulconazole could also have electrostatic interactions with the conserved D154, somewhat like the arginine P2 residue of the molecule co-crystallized with furin (7). In addition, the hydrophobic valine P3 residue of the inhibitor co-crystallized with furin would be here replaced by the hydrophobic 2,4 dichlorophenyl P3 moiety of Sulconazole (7). A binding score between Sulconazole and furin was re-computed after energy minization with the MolDock package (8) and found to be around −120 kcal/mol (dominated by favorable steric interactions with a small contribution from electrostatic interactions) and about −200 kcal/mol between furin and the modified peptide (the predicted score is better as the peptide is much larger than Sulconazole and there are more electrostatic interactions) while the score between FXa and Rivaroxaban using the same protocol was found to be around −152 kcal/mol. Scores between targets can not be directly compared, but by taking into account these values and the structural analysis mentioned above, it seems likely that Sulconazole inhibits furin. To evaluate the ability of Sulconazole to inhibit cellular PCs substrate maturation, we directly analyzed the cleavage of MT1-MMP using Western blot analysis. As illustrated in FIG. 1G, Sulconazole inhibits the cleavage of MT1-MMP, as assessed by the accumulation of its unprocessed form (63 KDa). Incubation of T cells with Sulconazole for 24 h repressed PD-1 expression in PMA/Io-activated cells at the RNA (FIG. 1H) and protein levels (FIGS. 1I and 1J). Immunofluorescence analysis of activated T cells treated with Sulconazole for 24 h show a 50% reduction of PD-1⁺ cells (FIGS. 1K and 1L). Similarly, NF—KB phosphorylation after PMA/Io stimulation was also inhibited (FIG. 1M). The use of JRT3 cells co-cultured with EPCR-expressing HT29 cancer cells, revealed that CD69 expression and number of CD69 positive cells were increased in the absence or presence of Sulconazole, while compared to JRT3 cells cultured alone (FIGS. 1N and 1O). These finding highlights the potential use of drug repositioning process for the identification of save furin inhibitor able to repress PD-1 expression in T cells.

### CONCLUSION

The use of drug repositioning allowed us to identify Sulconazole as a pharmological inhibitor of furin able to repress PD-1 expression in T cells. This strategy is gaining growing attention in the drug discovery field since it represents an effective way to exploit new molecular targets-related diseases. This approach capitalizes on the context that approved drugs and probably abandoned compounds have already been tested in humans and that all information on their pharmacology, and toxicity is available. Drug repositioning is also reinforced since conjoint molecular pathways are involved in different diseases. In addition, this strategy can significantly reduce the cost and development time since compounds that have demonstrated safety in humans it often omits the need for phase I clinical trials.

### REFERENCES

Throughout this application, various references describe the state of the art to which this invention pertains. The disclosures of these references are hereby incorporated by reference into the present disclosure.
1. Scamuffa N, Calvo F, Chrétien M, Seidah N G, Khatib A M. Proprotein convertases: lessons from knockouts. FASEB J. 2006; 20(12):1954-1963.
2. Scamuffa N, et al. Selective inhibition of proprotein convertases represses the metastatic potential of human colorectal tumor cells. J Clin Invest. 2008; 118(1):352-363.
3. Senzer N, et al. Phase i trial of bi-shRNAi furin/GMCSF DNA/autologous tumor cell vaccine (FANG) in advanced cancer. Mol Ther. 2012; 20(3):679-686.
4. Seidler J, McGovern S L, Doman T N, Shoichet B K. Identification and prediction of promiscuous aggregating inhibitors among known drugs. J Med Chem. 2003; 46(21):4477-4486.
5. Austin S, Taouji S, Chevet E, Wodrich H, Rayne F. Using Alphascreen® to identify small-molecule inhibitors targeting a conserved host-pathogen interaction. In: Methods in Molecular Biology. 2016:453-467.
6. Roehrig S et al. Discovery of the Novel Antithrombotic Agent 5-Chloro-N-({(5S)-2-oxo-3-2-carboxamide (BAY 59-7939): An Oral, Direct Factor Xa Inhibitor. J Med Chem. 2005; (Table 1):5900-5908.
7. Dahms S O, et al. X-ray structures of human furin in complex with competitive inhibitors. ACS Chem Biol. 2014; 9(5):1113-1118.
8. Thomsen R, Christensen M H. MolDock: A new technique for high-accuracy molecular docking. J Med Chem. 2006; 49(11):3315-3321.
9. Scior T, et al. Recognizing pitfalls in virtual screening: A critical review. J Chem Inf Model. 2012; 52(4):867-881.
10. Wishart D S, et al. DrugBank 5.0: A major update to the DrugBank database for 2018. Nucleic Acids Res. 2018; 46(D1):D1074-D1082.
11. Ursu O, et al. DrugCentral: Online drug compendium. Nucleic Acids Res. 2017; 45(D1):D932-D939.
12. Huang R, et al. The NCGC pharmaceutical collection: A comprehensive resource of clinically approved drugs enabling repurposing and chemical genomics. Sci Transl Med. 2011; 3(80). doi:10.1126/scitranslmed.3001862.

1. (canceled)

2. A method of treating a disease involving furin activity in a subject in need thereof comprising administering to the patient a therapeutically effective amount of Sulconazole.

3. The method of claim 2 wherein the disease involving furin activity is an autoimmune inflammatory disease.

4. The method of claim 2 wherein the disease involving furin activity is a viral infection.

5. The method of claim 2 wherein the disease involving furin activity is a neurodegenerative disease.

6. The method of claim 2 wherein the disease involving furin activity is a cancer.

7. A method of enhancing the proliferation, migration, persistence and/or activity of cytotoxic T lymphocytes (CTLs) in a subject in need thereof comprising administering to the subject a therapeutically effective amount of Sulconazole.

8. A method of therapy in subjects in need thereof, comprising administering to the subject a therapeutically effective amount of Sulconazole that reduces the expression of an immune checkpoint protein, wherein said administration enhances the proliferation, migration, persistence and/or activity of cytotoxic T lymphocytes (CTLs) in the subject.

9. A method of reducing T cell exhaustion in a subject in need thereof comprising administering to the subject a therapeutically effective amount of Sulconazole.

10. A method of treating cancer in a patient in need thereof comprising i) quantifying the density of cytotoxic T lymphocytes that express at least one immune checkpoint protein in a tumor tissue sample obtained from the patient ii) comparing the density quantified at step i) with a predetermined reference value and iii) administering to the patient a therapeutically effective amount of Sulconazole when the density quantified at step i) is higher than the predetermined reference value.

11. The method of claim 6 wherein Sulconazole is administered to the subject in combination with at least one immune checkpoint inhibitor.

12. The method of claim 11 wherein the immune checkpoint inhibitor is selected from the group consisting of PD-1 antagonists, PD-L1 antagonists, PD-L2 antagonists, CTLA-4 antagonists, VISTA antagonists, TIM-3 antagonists, LAG-3 antagonists, IDO antagonists, KIR2D antagonists, A2AR antagonists, B7-H3 antagonists, B7-H4 antagonists, and BTLA antagonists.

13. An in vitro or ex vivo method of reducing the expression of at least one immune checkpoint protein in a population of immune cells comprising contacting the population of T cells with an amount of Sulconazole sufficient to reduce the expression of the at least one immune checkpoint protein.

14. The method of claim 13 wherein the population of immune cells is a population of macrophages, monocytes or dendritic cells.

15. The method of claim 13 wherein the population of immune cells is a population of T cells, natural killer (NK) cells, or natural killer T (NKT) cells.

16. The method claim 15 wherein the population of T cells is a population of CAR-T cells.

17. The method of claim 10, wherein the at least one immune checkpoint protein is PD-1.

18. The method of claim 10 wherein Sulconazole is administered to the subject in combination with at least one immune checkpoint inhibitor.
